(19) United States
(12) Patent Application Publication
(10) Pub. No.: US 2019/0241917 A1
(43) Pub. Date: Aug. 8, 2019

(54) METHOD FOR PRODUCING AND SEPARATING LIPIDS

(71) Applicant: HOLIFERM LIMITED, Parkgate, Neston (GB)

(72) Inventors: James Benjamin WINTERBURN, Manchester (GB); Ben Michael DOLMAN, Manchester (GB)

(21) Appl. No.: 16/312,394

(22) PCT Filed: Apr. 28, 2017
(86) PCT No.: PCT/GB2017/051205

§ 371 Date: Dec. 21, 2018

(30) Foreign Application Priority Data
Jun. 22, 2016 (GB) 1610932.4

(51) Int. Cl. C12P 7/64; B01D 17/02; C12M 1/26; C12M 1/00; C12P 19/12

(52) U.S. Cl. CPC C12P 7/64; B01D 17/0214; C12P 19/12; C12M 33/22; C12M 47/10; C12P 7/6409

(57) ABSTRACT

There are provided methods for the production of lipids such as sophorolipids. Also provided are apparatus for use in the production.

### FIELD OF INVENTION

The present invention relates to the production of lipids, particularly, though not exclusively, to the production of sophorolipids and to apparatus for use in said production.

### BACKGROUND TO INVENTION

Microbial production of lipids and in particular glycolipids such as sophorolipids is known.

Sophorolipids consist of a hydrophilic sophorose disaccharide bound to a hydrophobic fatty acid with a typical chain length of 16-18 carbon atoms. The fatty acid may be joined by an ester bond to the second glucose monomer, giving a lactonic sophorolipid, or joined only to one glucose monomer, giving an acidic sophorolipid due to the unbound fatty acid. These and other differences in the fatty acid chain and acetylation of the sophorose molecules give a range of different structures and properties.

While several yeast strains are able to synthesize sophorolipids, most industrial use is focused on Candida bombicola ATCC 22214. Sophorolipid concentrations of over 300 g l⁻¹ at productivities of around 2 g l⁻¹ h⁻¹ are achievable in submerged C. bombicola fermentations, using vegetable oils and glucose as substrates.

Known lipid production such as sophorolipid production uses fed batch fermentation in a fermenter. Sophorolipid producing fermentations begin with a cell growth phase, which typically lasts until the nitrogen in the media is depleted, at which point the sophorolipid production rate increases significantly, if both a hydrophilic and hydrophobic carbon source are present. The sophorolipid production phase lasts for around 200 hours, at which point the dissolved oxygen level in the fermenter cannot be maintained due to oxygen mass transfer limitation. This is caused by the highly viscous nature of the sophorolipid produced, meaning the fermentation must be stopped and the sophorolipid recovered.

With known production methods the presence of a separate sophorolipid phase in the fermenter significantly reduces the oxygen mass transfer coefficient, kLa, by both providing a resistance to mass transfer across the air/liquid interface and increasing the viscosity of the medium, which results in oxygen limitation, increased stirring power requirements and non-homogeneity in the fermenter.

Accordingly, the present invention aims to address at least one disadvantage associated with the prior art whether discussed herein or otherwise.

### SUMMARY OF INVENTION

According to a first aspect of the present invention there is provided a method of producing lipids wherein the method comprises:
(a) performing a fermentation in a fermenter to produce a broth comprising lipid product;
(b) transferring broth comprising lipid product from the fermenter to a separator; (c) allowing a lipid phase comprising lipid product to separate from other constituents of the broth in the separator;
(d) returning broth having had lipid product separated therefrom from the separator to the fermenter; and
(e) transferring lipid product from the separator.

Suitably, the method comprises producing lipids selected from the group consisting of:
hydrocarbons, terpenoids, fats, oils, fatty acids, glycolipids and other compounds and which comprise molecules, produced by an organism, that are insoluble in water or amphiphilic and generally soluble in a non-polar solvent.

Suitably, the method comprises producing lipids selected from the group consisting of: hydrocarbons, terpenoids, fats, oils, fatty acids and glycolipids.

Suitably, the method comprises producing lipids selected from the group consisting of:

terpenoids, fats, oils, fatty acids and glycolipids.

Suitably, the method comprises a method of producing terpenoids. Suitably, the lipid product comprises terpenoids. The lipid product may consist of terpenoids.

Suitably, step (d) comprises returning broth having had a lipid phase comprising terpenoids separated therefrom from the separator to the fermenter. Suitably, step (e) comprises transferring a lipid phase comprising terpenoids from the separator. Suitably, step (e) comprises transferring a lipid product phase comprising terpenoids from the separator.

Suitably, the method comprises a method of producing glycolipids. Suitably, the lipid product comprises glycolipids. The lipid product may consist of glycolipids.

Suitably, step (d) comprises returning broth having had a lipid phase comprising glycolipids separated therefrom from the separator to the fermenter. Suitably, step (e) comprises transferring a lipid phase comprising glycolipids from the separator. Suitably, step (e) comprises transferring a lipid product phase comprising glycolipids from the separator.

Suitably, there is provided a method of producing lipids wherein the method comprises:
(a) performing a fermentation in a fermenter to produce a broth comprising glycolipids;
(b) transferring broth comprising glycolipids from the fermenter to a separator;
(c) allowing a lipid phase comprising glycolipids to separate from other constituents of the broth in the separator;
(d) returning broth having had glycolipids separated therefrom from the separator to the fermenter; and
(e) transferring glycolipids from the separator.

Suitably, the method comprises a method of producing lipids selected from sophorolipids, rhamnolipids and mannosylerythritol lipids. Suitably, the lipid product comprises one or more lipids selected from sophorolipids, rhamnolipids and mannosylerythritol lipids. The lipid product may consist of one or more lipids selected from sophorolipids, rhamnolipids and mannosylerythritol lipids.

Suitably, step (d) comprises returning broth having had a lipid phase comprising sophorolipids, rhamnolipids and/or mannosylerythritol lipids separated therefrom from the separator to the fermenter. Suitably, step (e) comprises transferring a lipid phase comprising sophorolipids, rhamnolipids and/or mannosylerythritol lipids from the separator. Suitably, step (e) comprises transferring a lipid product phase comprising sophorolipids, rhamnolipids and/or mannosylerythritol lipids from the separator.

Suitably, the method comprises a method of producing sophorolipids. Suitably, the lipid product comprises sophorolipids. The lipid product may consist of sophorolipids.

Suitably, step (d) comprises returning broth having had a lipid phase comprising sophorolipids separated therefrom from the separator to the fermenter. Suitably, step (e) comprises transferring a lipid phase comprising sophorolipids from the separator. Suitably, step (e) comprises transferring a lipid product phase comprising sophorolipids from the separator.

Suitably, there is provided a method of producing lipids wherein the method comprises:
(a) performing a fermentation in a fermenter to produce a broth comprising sophorolipids;
(b) transferring broth comprising sophorolipids from the fermenter to a separator;
(c) allowing a lipid phase comprising sophorolipids to separate from other constituents of the broth in the in the separator;
(d) returning broth having had sophorolipids separated therefrom from the separator to the fermenter; and
(e) transferring sophorolipids from the separator.

Suitably, steps (c), (d) and (e) are preformed concurrently with step (b). Suitably, step (b) is performed concurrently with step (a). Suitably, steps (b), (c), (d) and (e) are performed concurrently with step (a).

Steps (c), (d) and (e) may be performed as continuous steps. Steps (b), (c), (d) and (e) may be performed as continuous steps. Steps (a), (b), (c), (d) and (e) may be performed as continuous steps.

Steps (c), (d) and (e) may be operated intermittently but may suitably be performed as continuous steps during the periods for which they are operated. Steps (b), (c), (d) and (e) may be operated intermittently but may suitably be performed as continuous steps during the periods for which they are operated.

Suitably, the method comprises performing a multiple pass separation and suitably comprises performing steps (b), (c) and (d) in a continuous cycle.

Suitably, the method comprises performing a separation by continuously circulating broth from the fermenter, through the separator and back to the fermenter for a period of time whilst performing a fermentation in the fermenter. Suitably, said period of time is at least 30 minutes.

Suitably, the method comprises performing a separation by continuously circulating broth from the fermenter, through the separator and back to the fermenter for a plurality of periods of time during the duration of a fermentation with pauses between said periods. Alternatively, the method may comprise performing a separation by continuously circulating broth from the fermenter, through the separator and back to the fermenter without any pauses during a fermentation.

Suitably, the method comprises producing sophorolipids using Candida bombicola ATCC 22214.

Suitably, the method comprises circulating broth between the fermenter and separator over the duration of the fermentation. Suitably, the method comprises circulating broth between the fermenter and separator at intervals over the duration of the fermentation. Suitably, the method comprises continuously circulating broth between the fermenter and separator.

Suitably, the method comprises circulating broth between the fermenter and separator over a period of at least 1 hour. Suitably, the method comprises circulating broth between the fermenter and separator over a period of at least 10 hours, for example at least 20 hours. Suitably, the method comprises circulating broth between the fermenter and separator over a period of at least 50 hours, for example at least 100 hours. Suitably, the method comprises circulating broth between the fermenter and separator over a period of at least 150 hours, for example at least 200 hours.

Suitably, the method comprises transferring broth comprising lipid product from the fermenter, separating a lipid product from other constituents of the broth and returning broth having had a lipid product separated therefrom to the fermenter over the duration of the fermentation. Suitably, the method comprises continuously transferring broth comprising lipid product from the fermenter, separating a lipid product from other constituents of the broth and returning broth having had a lipid product separated therefrom to the fermenter. Suitably, the method comprises returning substrate lipids to the fermenter. The method may comprise returning lipid product to the fermenter as the separation in the separator may be incomplete and may separate some but not all lipid product from the broth that is then returned to the fermenter.

Suitably, the method comprises performing a fermentation in a fermenter and transferring broth comprising lipid product from the fermenter, separating a lipid product from other constituents of the broth and returning broth having had a lipid product separated therefrom to the fermenter over a period of at least 1 hour. The transfer, separation and return may be performed continuously or may be performed intermittently. Suitably, if performed intermittently the transfer, separation and return are performed on a plurality of occasions during the fermentation and are suitably operated with continuous re-circulation between the fermenter and separator during said occasions of operation.

Suitably, the method comprises transferring broth comprising lipid product from the fermenter, separating a lipid product from other constituents of the broth and returning broth having had a lipid product separated therefrom to the fermenter either continuously or on a plurality of occasions over a period of at least 10 hours, for example at least 20 hours. Suitably, the method comprises transferring broth comprising lipid product from the fermenter, separating a lipid product from other constituents of the broth and returning broth having had a lipid product separated therefrom to the fermenter either continuously or on a plurality of occasions over a period of at least 50 hours, for example at least 100 hours. Suitably, the method comprises transferring broth comprising lipid product from the fermenter, separating a lipid product from other constituents of the broth and returning broth having had a lipid product separated therefrom to the fermenter either continuously or on a plurality of occasions over a period of at least 150 hours, for example at least 200 hours.

Suitably, the method comprises operating a re-circulating separating process on a plurality of occasions, for example at least three occasions, over a period of fermentation which lasts at least 10 hours and wherein said separating process comprises transferring broth comprising lipid product from the fermenter, separating lipid product from other constituents of the broth and returning broth having had lipid product separated therefrom to the fermenter.

Suitably, the method comprises transferring broth from the fermenter and returning broth to the fermenter such that the broth in the fermenter comprises at least 50% by weight of the sum of the broth in the fermenter and separator. Suitably, the method comprises transferring broth from the fermenter and returning broth to the fermenter such that the broth in the fermenter comprises at least 60% by weight of the sum of the broth in the fermenter and separator, for example at least 70% by weight. Suitably, the method comprises transferring broth from the fermenter and returning broth to the fermenter such that the broth in the fermenter comprises at least 80% by weight of the sum of the broth in the fermenter and separator, for example at least 90% by weight. Suitably, the method comprises transferring broth from the fermenter and returning broth to the fermenter such that the broth in the fermenter comprises at least 95% by weight of the sum of the broth in the fermenter and separator, for example at least 98% by weight.

Suitably, the method comprises adding substrate to the fermenter after fermentation has been commenced. Suitably, the method comprises adding substrate to the fermenter over the duration of the fermentation. Suitably, the method comprises continuously adding substrate. Suitably, the substrate comprises lipids. Suitably, the substrate comprises lipids distinct from the lipid product to be produced by the method. Suitably, the method comprises adding oil and/or sugar to the fermenter after the fermentation has been commenced. Suitably, the method comprises adding oil and/or sugar to the fermenter over the duration of the fermentation. Suitably, the method comprises continuously adding oil and/or sugar to the fermenter.

Suitably, the method comprises adding vegetable oil to the fermenter, suitably rapeseed oil. Suitably, the method comprises adding glucose to the fermenter.

The method may comprise increasing the quantity of broth in the fermenter over time by adding oil and/or sugar to the broth.

Suitably, the method comprises ending fermentation with the fermenter at 60% or more of its capacity, for example at 70% or more of its capacity. The method may comprise ending fermentation with the fermenter at 80% or more of its capacity, for example at 90% or more. The method may comprise ending fermentation with the fermenter at 95% or more of its capacity.

Suitably, since broth is transferred from the fermenter to the separator and returned to the fermenter after lipids have been separated from the broth additional capacity is provided in the fermenter by the separation process. It may thus be possible to utilise a greater proportion of the fermenters capacity at the commencement of fermentation.

Suitably, the method comprises commencing fermentation with the fermenter at 50% or more of its capacity, for example at 60% or more. Suitably, the method comprises commencing fermentation with the fermenter at 70% or more of its capacity.

Suitably, the method comprises transferring broth comprising lipid product in a concentration of X g l⁻¹ of the removed broth from the fermenter to the separator and returning broth comprising lipid product in a concentration of Y g l⁻¹ of returned broth from the separator to the fermenter, wherein Y is less than X.

Suitably, the method comprises transferring broth comprising lipid product in a concentration of 50-250 g l⁻¹, for example in a concentration of 50-100 g l⁻¹, to the separator.

Suitably, the method comprises transferring broth comprising lipid product in a concentration of at least 5 g l⁻¹ to the separator, for example at least 10 g l⁻¹. Suitably, the method comprises transferring broth comprising lipid product in a concentration of at least 15 g l⁻¹ to the separator, for example at least 20 g l⁻¹. Suitably, the method comprises transferring broth comprising lipid product in a concentration of at least 50 g l⁻¹ to the separator, for example at least 80 g l⁻¹.

Suitably, the method comprises transferring broth comprising sophorolipids in a concentration of at least 5 g l⁻¹ to the separator, for example at least 10 g l⁻¹. Suitably, the method comprises transferring broth comprising sophorolipids in a concentration of at least 15 g l⁻¹ to the separator, for example at least 20 g l⁻¹. Suitably, the method comprises transferring broth comprising sophorolipids in a concentration of at least 50 g l⁻¹ to the separator, for example at least 80 g l⁻¹.

Suitably, the method comprises returning broth comprising lipid product in a concentration of less than 80 g l⁻¹ to the fermenter. The method may comprise returning broth comprising lipid product in a concentration of less than 50 g l⁻¹ to the fermenter, for example less than 20 g l⁻¹.

Suitably, the method comprises returning broth comprising sophorolipids in a concentration of less than 80 g l⁻¹ to the fermenter. The method may comprise returning broth comprising sophorolipids in a concentration of less than 50 g l⁻¹ to the fermenter, for example less than 20 g l⁻¹.

Suitably, the method comprises causing broth to have a residence time of at least 10 seconds in the separator. Suitably, the method comprises causing broth to have a residence time of at least 30 seconds in the separator, for example at least 60 seconds. Suitably, the method comprises causing broth to have a residence time of no more than 180 seconds, for example no more than 120 seconds in the separator. The method may comprise removing broth from the fermenter for the minimum time necessary to allow separation of lipid product from said broth.

Suitably, the method comprises maintaining the concentration of lipid product in the broth in the fermenter below 100 g l⁻¹, for example below 80 g l⁻¹.

Suitably, the method comprises maintaining the concentration of sophorolipids in the broth in the fermenter below 100 g l⁻¹, for example below 80 g l⁻¹.

Suitably, the method comprises agitating the broth in the fermenter. Suitably, the method comprises aerating the broth in the fermenter. Suitably, the method comprises stirring the broth in the fermenter.

Suitably, the method comprises maintaining the concentration of lipid product in the broth in the fermenter below 80 g l⁻¹ to keep the viscosity of the broth sufficiently low that reduced stirring of the broth is required.

Suitably, the method comprises separating lipid product from other constituents of the broth without stopping fermentation. Suitably the method comprises separating lipid product from the broth in the separator whilst fermentation continues in the fermenter and returning broth from the separator to the fermenter such that said broth can continue to be fermented after lipid product has been separated therefrom.

Suitably, step (a) is performed for a period of time before step (b) is commenced. Suitably, step (a) is continued whilst step (b) is performed.

The method may comprise performing step (a) and (b) until the broth in the separator begins to separate into a lipid phase comprising lipid product and a bulk broth phase. The method may comprise continuing step (a) and (b) after a lipid phase comprising lipid product separates from a bulk broth phase in the separator.

Suitably, the method comprises minimising or preventing the separation of the broth into phases in the fermenter. Suitably, the method comprises minimising or preventing the formation of a lipid phase in the broth in the fermenter. Suitably, the method comprises controlling the concentration of lipid product in the broth in the fermenter and agitating the broth in the fermenter to minimise or prevent the formation of a lipid phase in the fermenter.

Suitably, if a lipid phase forms in the broth in the fermenter said phase (fermenter lipid phase) comprises lipid product and other constituents.

Suitably, the method comprises agitating the broth in the fermenter. Suitably, the method comprises agitating the broth in the fermenter to mix lipids with other constituents of the broth. The method may comprise agitating the broth to mix a lipid phase with other constituents of the broth. The method may comprise mixing a fermenter lipid phase with a bulk broth phase in the fermenter to form a mixed broth.

Suitably, step (b) is commenced after lipid production has commenced in step (a). Suitably, step (b) is commenced before the broth in the fermenter begins to form a lipid phase and a bulk broth phase.

Suitably, if a lipid phase forms in the broth in the fermenter said fermenter lipid phase is mixed with a bulk broth phase in the fermenter to form a mixed broth and step (b) comprises transferring said mixture.

Suitably, step (b) comprises transferring lipid product together with other broth constituents from the fermenter.

Suitably, step (c) begins once step (b) is commenced. Suitably, step (b) is continued whilst step (c) is performed. Suitably, step (a) is continued whilst step (c) is performed. Suitably, step (d) is performed whilst step (b) is performed.

Suitably, step (c) comprises allowing the broth transferred from the fermenter to separate into phases. Step (c) may comprise causing the broth to separate into phases. Suitably, step (c) comprises a gravity separation step. Step (c) suitably comprises allowing the broth to settle into lighter and denser phases.

Suitably, the separation is performed as a re-circulating process. Suitably, the separation is performed as a dynamic rather than static process and thus may avoid leaving cells in hypoxic conditions for any significant length of time.

Suitably, the method comprises performing step (c) until a lipid phase forms in the broth in the separator due to settling whilst the broth in the fermenter continues to be mixed to prevent phase separation in the fermenter. Suitably, the method comprises continuing step (c) after a lipid phase forms in the broth in the separator. Suitably, the method comprises performing step (c) before a lipid phase forms in the broth in the fermenter.

Suitably, step (c) comprises allowing separation of the broth in the separator to provide a lipid phase comprising lipid product and a bulk broth comprising other constituents of the broth.

Suitably, the lipid phase in the broth in the separator comprises lipid product and other constituents. Suitably, the lipid phase comprises lipid product in a concentration of at least 20% by weight, for example at least 30% by weight. Suitably, the lipid phase comprises lipid product in a concentration of at least 40% by weight, for example at least 50% by weight. Suitably, the lipid phase comprises lipids and water.

Suitably, the lipid phase which separates from other constituents of the broth in the separator is a lipid product phase comprising lipid product. Suitably, the lipid product phase comprises lipid product and less than 10% by weight of other lipids.

Suitably, the method comprises separating broth in a separator into a lipid product phase comprising lipid product and a bulk broth phase. Suitably, the bulk broth phase comprises substrate lipids. Suitably, the lipid product phase comprises lipid product and less than 10% by weight of substrate lipids. Suitably, the lipid product phase is a glycolipid phase. Suitably, the lipid product phase is a sophorolipid phase.

Suitably, the lipid product phase comprises lipid product and water in a concentration of at least 80% by weight. Suitably, the lipid product phase comprises water and lipid product in a concentration of at least 90% by weight, for example 95% by weight.

Suitably, the bulk broth in the separator comprises substrate and/or cells. The bulk broth may comprise lipid product. Suitably, the bulk broth comprises lipid product in a concentration of no more than 20% by weight, for example no more than 15% by weight. Suitably, the bulk broth comprises lipid product in a concentration of no more than 10% by weight, for example no more than 5% by weight.

The lipid phase may be lighter or denser than the bulk broth depending on the conditions in the fermenter and/or the constituents of the broth. Suitably, the method comprises using a separator adapted such that the separator lipid phase can be transferred from the separator regardless of whether said separator lipid phase is more or less dense than the bulk broth. Suitably, the method comprises using a separator adapted such that broth having had a lipid phase separated therefrom (bulk broth) can be returned to the fermenter regardless of whether said lipid phase is more or less dense than said broth.

Suitably, the method comprises returning broth from the separator to the fermenter once step (b) is commenced. The method may thus comprise returning broth that has not had a lipid phase separated therefrom to the fermenter until step (c) has proceeded for sufficient time to allow a lipid phase to separate.

Suitably, step (d) is commenced at substantially the same time as step (c) is commenced. Suitably, step (d) is commenced at substantially the same time as step (b) is commenced. Suitably, step (c) is continued whilst step (d) is performed. Suitably, step (b) is continued whilst step (d) is performed. Suitably, step (a) is continued whilst step (d) is performed.

Suitably, step (e) is commenced a period of time after a lipid phase has first separated from other constituents of the broth in step (c). Suitably, step (c) is continued whilst step (e) is performed. Suitably, step (b) is continued whilst step (e) is performed. Suitably, step (a) is continued whilst step (e) is performed.

Step (d) may be commenced prior to step (e). Suitably, step (d) is continued whilst step (e) is performed.

Suitably, the method comprises maintaining production conditions to provide a lipid phase that can be separated from the broth by gravity separation in the separator. Suitably, the method comprises performing separation using a separator comprising a settling column.

Suitably, the method comprises maintaining the pH of the broth in the fermenter at between pH 2 and pH 5, for example between pH 2.5 and pH 4.5. Suitably, the method comprises maintaining the pH of the broth in the fermenter at between pH 3.0 and pH 4.0, for example at pH 3.5.

Suitably, the method comprises maintaining the dissolved oxygen level of the broth in the fermenter at 20% or greater, for example at 25% or greater. Suitably, the method comprises maintaining the dissolved oxygen level of the broth in the fermenter at between 20% and 40%, for example between 25% and 35%. Suitably, the method comprises maintaining the dissolved oxygen level of the broth in the fermenter at 30%.

Suitably, the method comprises feeding sugar, suitably glucose. Suitably, the method comprises feeding sugar, suitably glucose, at a rate of at least 0.5 g l⁻¹ h⁻¹. The method may comprise feeding sugar, suitably glucose, at a rate of 1.0 g l⁻¹ h⁻¹ to 2.0 g l⁻¹ h⁻¹, for example 1.5 g l⁻¹ h⁻¹.

The method may comprise feeding vegetable oil, suitably rapeseed oil. Alternatively, or in addition the method may comprise feeding vegetable oil esters or oleic acid. Suitably, the method comprises feeding vegetable oil, suitably rapeseed oil, at a rate of at least 0.5 g l⁻¹ h⁻¹. The method may comprise feeding vegetable oil, suitably rapeseed oil, at a rate of 1.2 g l⁻¹ h⁻¹ to 2.2 g l⁻¹ h⁻¹, for example 1.7 g l⁻¹ h⁻¹.

Suitably, the method comprises separating a sophorolipid phase comprising at least 300 g l⁻¹ of sophorolipid, for example at least 400 g l⁻¹ of sophorolipid. Suitably, the method comprises separating a sophorolipid phase comprising at least 500 g l⁻¹ of sophorolipid, for example 550 g l⁻¹ sophorolipid.

Suitably, the method comprises separating a sophorolipid phase comprising cells at a concentration of 10% or less of the concentration of cells in the broth. Suitably, the method comprises separating a sophorolipid phase comprising 1 g l⁻¹ or less of cells. Suitably, the method comprises separating a sophorolipid phase comprising 0.1 g l⁻¹ or less of cells. Suitably, the method comprises separating a sophorolipid phase comprising substantially no cells.

Suitably, the method comprises returning broth comprising less than 80 g l⁻¹ of sophorolipids, to the fermenter. The method may comprise returning broth comprising less than 50 g l⁻¹ of sophorolipids, for example less than 30 g l⁻¹ of sophorolipids to the fermenter.

Suitably, the method comprises removing sophorolipids in the separator such that the broth in the fermenter is maintained with a level of less than 100 g l⁻¹ of sophorolipids, for example less than 80 g l⁻¹ of sophorolipids. The method may comprise removing sophorolipids in the separator such that the broth in the fermenter is maintained with a level of less than 50 g l⁻¹ of sophorolipids.

Suitably, the method comprises removing a viscous sophorolipid phase such that the viscosity of the broth in the fermenter is maintained at a lower viscosity than if the sophorolipid phase was not removed. Suitably, the method comprises removing a viscous sophorolipid phase such that a lower stirrer speed is required to maintain the desired oxygen level than would otherwise be the case.

Suitably, the method comprises reducing the volume requirement of a fermenter by removing a sophorolipid phase. Suitably, the method comprises reducing the fermenter volume requirement by 5% or more, for example by 10% or more. Suitably, the method comprises reducing the fermenter volume requirement by 20% or more, for example by 30% or more. The method may comprise reducing the fermenter volume requirement by up to 40%.

The method may comprise increasing productivity of a fermentation by removing a sophorolipid phase. The method may comprise increasing productivity of a fermentation by increasing available volume of a fermenter. The method may comprise increasing productivity by 5% or more, for example by 10% or more. The method may comprise increasing productivity by 20% or more, for example by 30% or more.

The method may comprise facilitating improved mixing of broth in the fermenter by removing a sophorolipid phase.

The method may comprise facilitating even distribution of dissolved oxygen in broth in the fermenter by removing a sophorolipid phase.

The method may comprise reducing agitation requirements and thus energy costs by removing a sophorolipid phase. The method may comprise reducing energy requirements for agitation by 20% or more, for example by 30% or more, for example by 40%.

The method may comprise allowing a fermentation to be performed for longer than would otherwise be the case by removing a sophorolipid phase.

Suitably, the method comprises using a gravity separator. Suitably, the method comprises using a separator comprising a settling column.

Suitably, the method comprises using a separator according to a second aspect of the present invention.

Suitably, the method comprises using an apparatus according to a third aspect of the present invention.

According to a second aspect of the present invention there is provided a separator adapted to separate a lipid phase from other constituents of a broth comprising lipids wherein the separator comprises:
(I) a separating chamber in which, in use, said broth can be allowed to reside for a period of time such that a lipid phase comprising lipid product separates from other constituents of the broth;
(II) a broth inlet to the separating chamber for transferring broth comprising lipid product into the separating chamber, in use; and
(III) outlets from the separating chamber for: (i) transferring broth having had lipid product separated therefrom from the separating chamber, in use; and (ii) transferring lipid product from the separating chamber, in use.

Suitably, the separator comprises two outlets (III). Suitably, the separator comprises three outlets (III). Suitably, the separator comprises three outlets (III) and is configured such that at least one outlet (III) can be selectively used as follows:
(i) for transferring broth having had lipid product separated therefrom from the separating chamber;
(ii) for transferring lipid product from the separating chamber; and
(iii) not used or used for pressure relief.

Suitably, the separator comprises a first outlet (IIIa) and a second outlet (IIIb). Suitably, the separator comprises a first outlet (IIIa), a second outlet (IIIb) and a third outlet (IIIc). Suitably, the separator comprises three outlets from the separating chamber and is configured such that, in use, one outlet is selectively not used during a separation.

Suitably, the separator comprises outlets (IIIa), (IIIb) and (IIIc) configured, to be selectively used as follows:
1. (IIIa) not used or used for pressure relief;
   (IIIb) for transferring a lipid product from the separating chamber;
   (IIIc) for transferring broth having had a lipid product removed therefrom from the separating chamber; or
2. (IIIa) for transferring a lipid product from the separating chamber;
   (IIIb) for transferring broth having had a lipid product removed therefrom from the separating chamber;
   (IIIc) not used or used for pressure relief.

Suitably, the separator comprises outlets (IIIa), (IIIb) and (IIIc) configured, to be selectively used as follows:
1. (IIIa) used for pressure relief;
   (IIIb) for transferring a lipid product from the separating chamber;
   (IIIc) for transferring broth having had a lipid product removed therefrom from the separating chamber; or
2. (IIIa) for transferring a lipid product from the separating chamber;
   (IIIb) for transferring broth having had a lipid product removed therefrom from the separating chamber;
   (IIIc) not used.

Suitably, the separator comprises two outlets which can be used as lipid product outlets and is adapted such that, in use, one of said outlets is selectively used for transferring a lipid product from the separating chamber.

Suitably, the separator comprises two outlets which can be used as broth outlets and is adapted such that, in use, one of said outlets is selectively used for transferring broth from the separating chamber.

Suitably, the separator comprises an outlet which can be used as a lipid product outlet located toward the upper, in use, end of the separating chamber. Suitably, the separator comprises an outlet which can be used as a lipid product outlet located toward the lower, in use, end of the separating chamber.

Suitably, there is provided a separator adapted to separate a lipid phase from other constituents of a broth comprising lipids wherein the separator comprises:
(I) a separating chamber in which, in use, said broth can be allowed to reside for a period of time such that a lipid phase comprising lipid product separates from other constituents of the broth;
(II) a broth inlet for transferring broth comprising lipid product into the separating chamber, in use;
(IIIa) a first outlet located toward the upper, in use, end of the separating chamber;
(IIIb) a second outlet located toward the lower, in use, end of the separating chamber; and
(IIIc) a third outlet located toward the lower, in use, end of the separating chamber.

Suitably, said first and second outlets (IIIa), (IIIb) can selectively be used to transfer a lipid product from the separating chamber, in use, depending on the density of the lipid phase comprising the lipid product.

Suitably, said second and third outlets (IIIb), (IIIc) can selectively be used to transfer broth having had lipid product removed therefrom from the separating chamber, in use, depending on the density of the lipid phase.

Suitably, the separating chamber (I) comprises an elongate chamber.

Suitably, the separating chamber comprises a settling column.

Suitably, the separating chamber is adapted such that, in use, the longitudinal axis of the chamber lies at between 10 degrees and 60 degrees to the horizontal, for example between 20 degrees and 40 degrees to the horizontal. Suitably, the separating chamber is adapted such that, in use, the longitudinal axis of the chamber lies at between 25 degrees and 30 degrees to the horizontal, for example at 30 degrees to the horizontal.

Suitably, the separating chamber comprises a cylindrical section. Suitably, the separating chamber comprises a cylindrical section for at least 90% of its length.

The separating chamber (I) may have a length at least 3 times its diameter, for example at least 4 times. The separating chamber may have a length at least 5 times its diameter, for example at least 6 times.

Suitably, the separating chamber has a frustoconical first, upper, in use, end. Suitably, the separating chamber has a flat base at its second, lower, in use, end.

Suitably, the broth inlet (II) and outlets (IIIb), (IIIc) are located at or towards opposed ends of the separating chamber. Suitably, the broth inlet (II) is located at or towards a first, upper, in use, end of the separating chamber and the outlets (IIIb), (IIIc) are located at or towards a second, lower, in use end of the separating chamber.

Suitably, the separator is configured such that, in use, broth has a residence time of at least 30 seconds in the separating chamber from entering the chamber through broth inlet (II) before exiting through an outlet (IIIb), (IIIc). Suitably, the separator is configured such that, in use, broth has a residence time of at least 60 seconds therein. Suitably, the separator is configured such that, in use, broth has a residence time of at least 90 seconds therein.

Suitably, the separator is configured such that, in use, broth has a residence time of no more than 180 seconds in the separating chamber from entering the chamber through broth inlet (II) before exiting through an outlet (IIIb), (IIIc). Suitably, the separator is configured such that, in use, broth has a residence time of no more than 150 seconds therein. Suitably, the separator is configured such that, in use, broth has a residence time of no more than 120 seconds therein.

Suitably, the broth inlet (II) is configured to transfer a broth comprising lipid product from a fermenter to the separator chamber. The broth inlet (II) may comprise a valve. The broth inlet (II) may comprise a conduit for connecting to a fermenter. The broth inlet (II) may comprise a pump for pumping broth from a fermenter into the separator chamber.

Suitably, the broth inlet (II) comprises an opening to the separating chamber towards an upper, in use, end of said separating chamber. Suitably, the broth inlet (II) comprises an opening to the separating chamber at an upper, in use, end of said separating chamber.

Suitably, the broth inlet (II) comprises an opening to the separating chamber which lies in an upper, in use, end wall of said separating chamber, suitably in a central region of said end wall. Suitably, the broth inlet (II) comprises an opening to the separating chamber which lies on the longitudinal axis of said separating chamber.

Suitably, the first outlet (IIIa) is configured, in use, to transfer a lipid phase comprising a lipid product from the separating chamber when said lipid phase has a lower density than other constituents of the broth and floats towards the top of the separating chamber.

The first outlet (IIIa) may comprise a valve. The first outlet (IIIa) may comprise a conduit for connecting to a collecting vessel or processing apparatus. The first outlet (IIIa) may comprise a pump for pumping a lipid product from the separating chamber.

Suitably, the first outlet (IIIa) comprises an opening to the separating chamber towards an upper, in use, end of said separating chamber. Suitably, the first outlet (IIIa) comprises an opening to the separating chamber in the upper, in use, 10% of the longitudinal length of said separating chamber.

Suitably, the first outlet (IIIa) comprises an opening to the separating chamber which lies in a side wall of said separating chamber, suitably in a region of said side wall toward the upper, in use, end of the chamber. Suitably, the first outlet (IIIa) comprises an opening to the separating chamber which lies off centre from the longitudinal axis of said separating chamber. Suitably, the first outlet (IIIa) comprises an opening to the separating chamber which lies above the longitudinal axis of said separating chamber, in use.

The second outlet (IIIb) may be configured, in use, to transfer a lipid phase comprising a lipid product from the separating chamber when said lipid phase has a higher density than other constituents of the broth and sinks towards the bottom of the separating chamber.

The second outlet (IIIb) may be configured, in use, to transfer broth having had lipid product removed therefrom from the separating chamber to a fermenter when a lipid phase comprising said lipid product has a lower density than other constituents of the broth and floats towards the top of the separating chamber.

The second outlet (IIIb) may comprise a valve. The second outlet (IIIb) may comprise a conduit for connecting to a collecting vessel or processing apparatus or fermenter. The second outlet (IIIb) may comprise a pump for pumping a lipid product or a broth having had lipid product removed therefrom from the separating chamber.

Suitably, the second outlet (IIIb) comprises an opening to the separating chamber towards a lower, in use, end of said separating chamber. Suitably, the second outlet (IIIb) comprises an opening to the separating chamber in the lower, in use, 10% of the longitudinal length of said separating chamber.

Suitably, the second outlet (IIIb) comprises an opening to the separating chamber which lies in a side wall of said separating chamber, suitably in a region of said side wall towards a lower, in use, end of the separating chamber. Suitably, the second outlet (IIIb) comprises an opening to the separating chamber which lies off centre from the longitudinal axis of said separating chamber. Suitably, the second outlet (IIIb) comprises an opening to the separating chamber which lies below the longitudinal axis of said separating chamber.

The third outlet (IIIc) may be configured, in use, to transfer a broth having had lipids removed therefrom from the separator chamber to a fermenter.

The third outlet (IIIc) may be configured, in use, to transfer broth having had lipid product removed therefrom from the separating chamber to a fermenter when a lipid phase comprising said lipid product has a higher density than other constituents of the broth and sinks towards the bottom of the separating chamber.

The third outlet (IIIc) may comprise a valve. The third outlet (IIIc) may comprise a conduit for connecting to a fermenter. The third outlet (IIIc) may comprise a pump for pumping broth having had lipid product removed therefrom from the separator to a fermenter.

Suitably, the third outlet (IIIc) comprises an opening to the separating chamber towards a lower, in use, end of said separating chamber. Suitably, the third outlet (IIIc) comprises an opening to the separating chamber in the lower, in use, 10% of the longitudinal length of said separating chamber.

Suitably, the third outlet (IIIc) comprises an opening to the separating chamber which lies in a side wall of said separating chamber, suitably in a region of said side wall toward the lower, in use, end of the separating chamber. Suitably, the third outlet (IIIc) comprises an opening to the separating chamber which lies off centre from the longitudinal axis of said separating chamber. Suitably, the third outlet (IIIc) comprises an opening to the separating chamber which lies above the longitudinal axis of said separating chamber, in use.

Suitably, the broth inlet (II) and third outlet (III) are configured to be in fluid communication with the same fermenter.

Suitably, the separator is adapted to separate from a broth lipids selected from the group consisting of: hydrocarbons, terpenoids, fats, oils, fatty acids, glycolipids and other compounds and which comprise molecules, produced by an organism, that are insoluble in water or amphiphilic and generally soluble in a non polar solvent.

Suitably, the separator is adapted to separate from a broth lipids selected from the group consisting of: hydrocarbons, terpenoids, fats, oils, fatty acids and glycolipids.

Suitably, the separator is adapted to separate from a broth lipids selected from the group consisting of: terpenoids, fats, oils, fatty acids and glycolipids.

Suitably, the separator comprises a separator for separating terpenoids from a broth.

Suitably, the separator comprises a separator for separating glycolipids from a broth. Suitably, the separator comprises a separator for separating sophorolipids from a broth.

Suitably, the separator is configured for use in a method according to the first aspect.

Suitably, the separator comprises two outlets (III). Suitably, the separator comprises two outlets (III) and is configured such that each outlet (III) can be selectively used as follows:
(i) for transferring broth having had lipid product separated therefrom from the separating chamber; and
(ii) for transferring lipid product from the separating chamber.

Suitably, there is provided a separator adapted to separate a lipid phase from other constituents of a broth comprising lipids wherein the separator comprises:
(I) a separating chamber in which, in use, said broth can be allowed to reside for a period of time such that a lipid phase comprising lipid product separates from other constituents of the broth;
(II) a broth inlet located toward a first end of the separating chamber for transferring broth comprising lipid product into the separating chamber, in use;
(IIIA) a first outlet located toward a second end of the separating chamber; and
(IIIB) a second outlet located toward a second end of the separating chamber.

Suitably, the broth inlet (II) and outlets (IIIA), (IIIB) are located at or towards opposed ends of the separating chamber. Suitably, the broth inlet (II) is located at or towards a first, lower, in use, end of the separating chamber and the outlets (IIIA), (IIIB) are located at or towards a second, upper, in use end of the separating chamber.

Suitably, the broth inlet (II) comprises an opening to the separating chamber at a lower, in use, end of said separating chamber. Suitably, the broth inlet (II) comprises an opening to the separating chamber which lies in a lower, in use, end wall of said separating chamber, suitably in a central region of said end wall. Suitably, the broth inlet (II) comprises an opening to the separating chamber which lies on the longitudinal axis of said separating chamber.

The first outlet (IIIA) may be configured, in use, to transfer broth having had lipid product removed therefrom from the separating chamber to a fermenter when a lipid phase comprising said lipid product has a lower density than other constituents of the broth and floats towards the top of the separating chamber.

Suitably, the first outlet (IIIA) comprises an opening to the separating chamber towards an upper, in use, end of said separating chamber. Suitably, the first outlet (IIIA) comprises an opening to the separating chamber in the upper, in use, 10% of the longitudinal length of said separating chamber.

Suitably, the first outlet (IIIA) comprises an opening to the separating chamber which lies in a side wall of said separating chamber, suitably in a region of said side wall towards an upper, in use, end of the separating chamber. Suitably, the first outlet (IIIA) comprises an opening to the separating chamber which lies off centre from the longitudinal axis of said separating chamber. Suitably, the first outlet (IIIA) comprises an opening to the separating chamber which lies below the longitudinal axis of said separating chamber, in use.

The second outlet (IIIB) may be configured, in use, to transfer lipid product for collection when a lipid phase comprising said lipid product has a lower density than other constituents of the broth and floats towards the top of the separating chamber.

Suitably, the second outlet (IIIB) comprises an opening to the separating chamber towards an upper, in use, end of said separating chamber. Suitably, the second outlet (IIIB) comprises an opening to the separating chamber in the upper, in use, 10% of the longitudinal length of said separating chamber.

Suitably, the second outlet (IIIB) comprises an opening to the separating chamber which lies in a side wall of said separating chamber, suitably in a region of said side wall toward the upper, in use, end of the separating chamber. Suitably, the second outlet (IIIB) comprises an opening to the separating chamber which lies off centre from the longitudinal axis of said separating chamber. Suitably, the second outlet (IIIB) comprises an opening to the separating chamber which lies above the longitudinal axis of said separating chamber, in use.

According to a third aspect of the present invention there is provided an apparatus for producing lipids, said apparatus comprising a fermenter having a fermentation chamber and a separator having a separating chamber and wherein said fermentation chamber and separating chamber are in fluid communication such that, in use, broth comprising lipid product can be transferred from the fermentation chamber to the separating chamber and broth having had lipid product separated therefrom can be transferred from the separating chamber to the fermentation chamber.

Suitably, the separator comprises a gravity separator. Suitably, the apparatus comprises a gravity separator as the only separator.

Suitably, the apparatus comprises a separator according to the second aspect. Suitably, the apparatus comprises a separator according to the second aspect as the only separator.

Suitably, the fermenter comprises an agitator. Suitably, the fermenter comprises a stirrer.

Suitably the fermenter comprises a sparger.

Suitably, there is provided an apparatus for producing lipids, said apparatus comprising a fermenter having a fermentation chamber in which lipids can be produced in a broth by fermentation and a separator having a separating chamber and wherein said fermentation chamber comprises an outlet in fluid communication with an inlet of the separating chamber and an inlet in fluid communication with an outlet of the separating chamber and wherein the apparatus is adapted such that, in use the separating chamber receives broth comprising lipid product from the fermenter, separates a lipid phase comprising lipid product from the broth and returns broth having had lipid product removed therefrom to the fermentation chamber.

Suitably the fermenter further comprises a feed inlet to allow substrate to be fed into the broth in the fermenter during fermentation. Suitably, the fermenter comprises a feed inlet for sugar, suitably glucose and vegetable oil, suitably rapeseed oil.

Suitably, the separator further comprises an outlet for transferring the lipid product from the separator and which may be in fluid communication with a collection vessel.

Suitably, the apparatus is adapted to produce lipids selected from the group consisting of: hydrocarbons, terpenoids, fats, oils, fatty acids, glycolipids and other compounds and which comprise molecules, produced by an organism, that are insoluble in water or amphiphilic and generally soluble in a non-polar solvent.

Suitably, the apparatus is adapted to produce lipids selected from the group consisting of: hydrocarbons, terpenoids, fats, oils, fatty acids and glycolipids.

Suitably, the apparatus is adapted to produce lipids selected from the group consisting of: terpenoids, fats, oils, fatty acids and glycolipids.

Suitably, the apparatus comprises an apparatus for producing terpenoids.

Suitably, the apparatus comprises an apparatus for producing glycolipids. Suitably, the apparatus comprises an apparatus for producing sophorolipids.

Suitably, the apparatus is configured for use in a method according to the first aspect.

### BRIEF DESCRIPTION OF DRAWINGS

The present invention will now be illustrated by way of example with reference to the accompanying drawings in which:

FIG. 1 shows a separator;

FIG. 2 shows an apparatus comprising a fermenter and separator;

FIG. 3 shows the apparatus of FIG. 2 in an alternative configuration;

FIG. 4 is a graph showing feeding of substrate for fermentations;

FIG. 5 is a graph showing feeding of substrate for a fermentation;

FIG. 6 is a graph showing stirrer speed and dissolved oxygen;

FIG. 7 shows an alternative embodiment of a separator;

FIG. 8 is a graph showing concentrations and lipid production for a fermentation; and

FIG. 9 is a graph showing substrate feeding for the fermentation of FIG. 8.

### DETAILED DESCRIPTION OF PREFERRED EMBODIMENTS

Example lipid productions were performed using a fermenter and separator to produce a broth comprising sophorolipids (lipid product) and to separate a sophorolipid phase from the broth (Examples 1 and 2). For comparison, a production of sophorolipids was performed without using a separator (Comparative Example 1C). A further example lipid production was performed using a fermenter and an alternative separator (Example 3).

Apparatus

For each of Examples 1 and 2 and Comparative Example 1C, the fermenter used was an Electrolab Fermac 320 fermentation system (Electrolab, UK) with a 2 l maximum working volume, H:D of 2, and an initial working volume of 1 l was used.

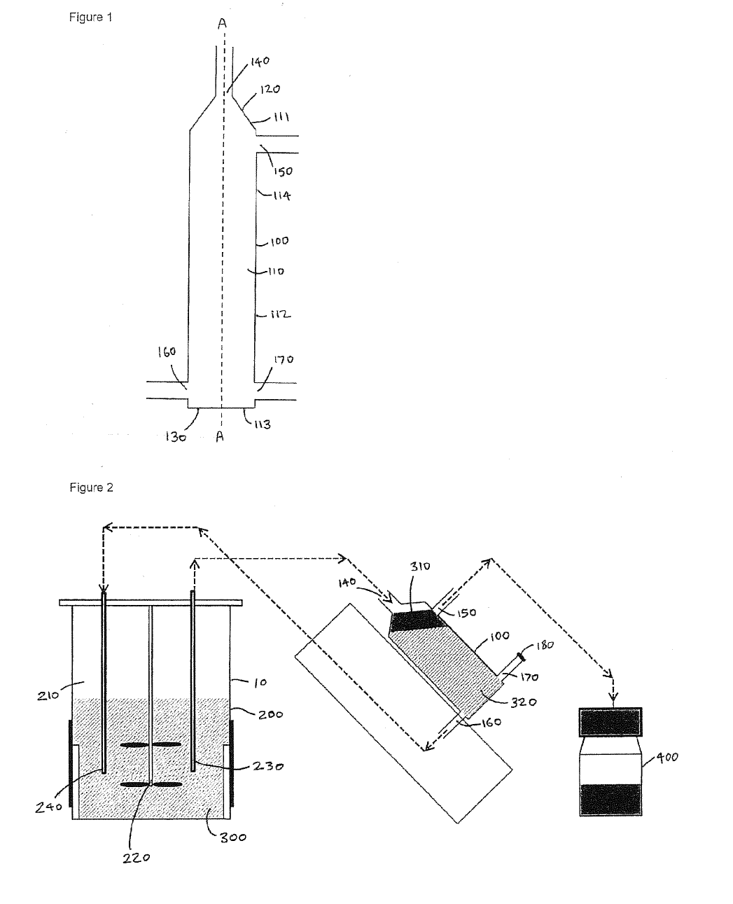

For Examples 1 and 2 the separator used was an in house built settling column illustrated in FIG. 1.

The separator 100 comprises a separating chamber 110. At the first, upper, in use, end 120 of the separator 100 the separating chamber 110 has a frusto conical section 111. The separating chamber 110 further comprises a cylindrical section 112 having a diameter of 50 mm and a length of 150 mm. The base 113 of the cylindrical section forms the lower, in use, end 130 of the separator.

The separator 100 comprises a broth inlet 140 and has an opening to the separating chamber 110 located on the longitudinal axis A-A of the separating chamber 110 at the first end 120 of the separator 100.

The separator comprises a first outlet 150, a second outlet 160 and a third outlet 170.

The first outlet 150 is located toward the first end 120 of the separator 100 and has an opening to the separating chamber 110 through a side wall 114 of the separating chamber 110. In use, the separator 100 is oriented such that the first outlet 150 lies above the longitudinal axis A-A of the separating chamber 110. Depending on the density of a lipid phase separated from a broth in the separator 100, in use, the first outlet 150 can be used to transfer a lipid product from the separator 100 or alternatively can be used for pressure relief.

The second outlet 160 is located toward the second end 130 of the separator 100 and has an opening to the separating chamber 110 through a side wall 114 of the separating chamber 110. In use, the separator 100 is oriented such that the second outlet 160 lies below the longitudinal axis A-A of the separating chamber 110. Depending on the density of a lipid phase separated from a broth in the separator 100, in use, the second outlet 160 can be used to transfer a lipid product from the separator 100 or alternatively can be used to transfer a broth having had lipid product separated therefrom from the separator 100.

The third outlet 170 is located toward the second end 130 of the separator 100 and has an opening to the separating chamber 110 through a side wall 114 of the separating chamber 110. In use, the separator 100 is oriented such that the second outlet 170 lies above the longitudinal axis A-A of the separating chamber 110. Depending on the density of a lipid phase separated from a broth in the separator 100, in use, the third outlet 170 can be used to transfer a broth having had lipid product separated therefrom from the separator 100 or alternatively can be redundant.

The third outlet 170 is positioned on the circumference of the cylinder 180 degrees from the second outlet 160 at the same distance from the end wall 113 of the separating chamber 110. The third outlet 170 is positioned on the circumference of the cylinder 0 degrees from the first outlet 150 but at the opposite end of the cylindrical section 112.

The separating chamber 110 is attached to a stand (not shown) which allows the angle of the longitudinal axis A-A of the separating chamber 110 relative to the horizontal to be varied. For Examples 1 and 2 the angle used was 30 degrees to the horizontal.

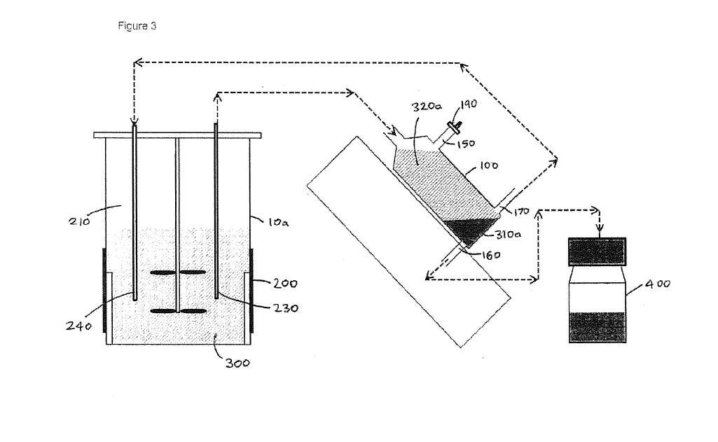

Apparatus 10 comprising a fermenter 200 and separator 100 configured as used for Example 1 is illustrated in FIG. 2. The separator was configured to separate a sophorolipid phase (lipid product phase) that was less dense than the broth.

The separator 100 comprises a separator according to FIG. 1.

The apparatus 10 is configured for the separator 100 to receive broth 300 comprising lipid product from the fermenter and to separate a lipid product phase 310 which is denser than a broth 300 from said broth to provide broth 320 having had the lipid product separated therefrom. The apparatus is further configured for the lipid product phase 310 to be transferred by a pump (not shown) and collected in a container 400 and the broth 320 having had the lipid product separated therefrom to be returned to the fermenter 200.

The separator 100 is angled such that the longitudinal axis A-A of the separating chamber 110 lies at 30 degrees to the horizontal. The first outlet 150 serves as an outlet for the lipid product phase 310, the second outlet 160 serves as an outlet for broth 320 having had the lipid product separated therefrom and the third outlet 170 is sealed with a stopper 180 and not used.

The fermenter 200 comprises a fermentation chamber 210 for holding broth 300. The fermenter 200 also comprises a stirrer 220 for agitating the broth. The fermenter also comprises an air sparger (not shown) for aerating the broth. The fermenter 200 further comprises a broth outlet 230 in fluid communication with a broth inlet 140 of the separator via a pump (not shown). In addition the fermenter comprises a broth inlet 240 in fluid communication via a pump (not shown) with the second outlet 160 of the separator 100.

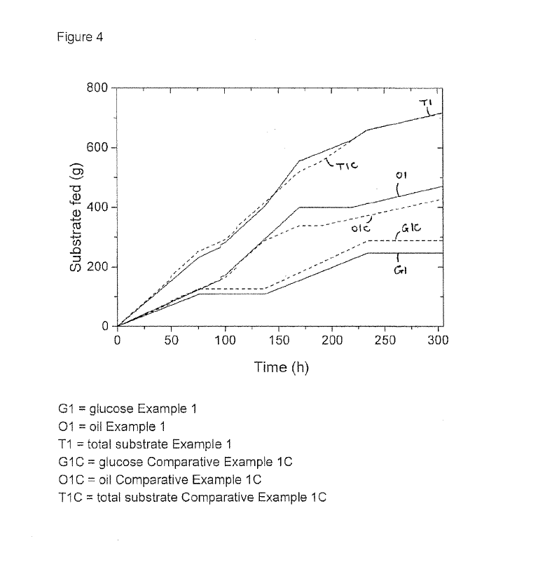

Apparatus 10a comprising a fermenter 200 and separator 100 configured as used for Example 2 is illustrated in FIG. 3. The separator was configured to separate a sophorolipid phase (lipid product phase) that was denser than the broth.

The apparatus 10a is substantially the same as apparatus 10 and like parts are numbered accordingly. The main difference between the configuration of apparatus 10a and apparatus 10 is in the use of the outlets 150, 160, 170 and the fluid communication between the separator 100 and fermenter 200.

The separator 100 comprises a separator according to FIG. 1.

The apparatus 10a is configured for the separator 100 to receive broth 300 comprising lipid product from the fermenter and to separate a lipid product phase 310a which is less dense than a broth 300a from said broth to provide broth 320a having had the lipid product separated therefrom. The apparatus is further configured for the lipid product phase 310a to be transferred by a pump (not shown) and collected in a container 400 and for the broth 320a having had the lipid product separated therefrom to be returned to the fermenter 200.

The separator 100 is angled such that the longitudinal axis A-A of the separating chamber 110 lies at 30 degrees to the horizontal. The first outlet 150 is used as a pressure relief outlet and provided with an air filter 190, the second outlet 160 serves as an outlet for the lipid product phase 310a and the third outlet 170 serves as an outlet for the broth 320a having had the lipid product separated therefrom.

The fermenter 200 comprises a fermentation chamber 210 for holding broth 300a. The fermenter 200 also comprises a stirrer 220 for agitating the broth. The fermenter also comprises an air sparger (not shown) for aerating the broth. The fermenter 200 further comprises a broth outlet 230 in fluid communication with a broth inlet 140 of the separator via a pump (not shown). In addition the fermenter comprises a broth inlet 240 in fluid communication via a pump (not shown) with the third outlet 170 of the separator 100.

Fermentations

For each of Examples 1 and 2 and Comparative Example 1C, the sophorolipid productions were fed batch fermentations using C. bombicola ATCC 22214.

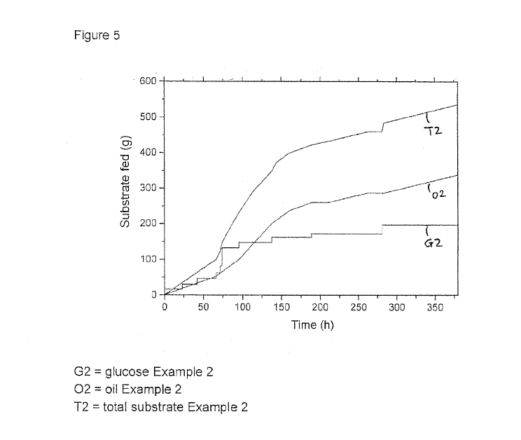

The substrates fed for the fermentations were glucose and rapeseed oil. The feeding rates of substrates were different for Example 1 and Example 2 so as to give a sophorolipid phase that separated to the top of the separator in Example 1 and to the bottom of the separator in Example 2. The substrates were fed at substantially the same rate for Comparative Example 1C as for Example 1. FIG. 4 shows the feeding of substrates for Example 1 and Comparative Example 10 and FIG. 5 shows the feeding of the substrates for Example 2.

For each of Examples 1 and 2 and Comparative Example 10 the growth medium for the fermentations, preculture and agar plates contained 6 g l⁻¹ yeast extract and 5 g l⁻¹ peptone. The initial concentration of glucose in all fermentations, preculture and agar plates was 100 g l⁻¹, with an initial rapeseed oil concentration of 50 g l⁻¹ in the fermenters, 100 g l⁻¹ in the preculture and 0 in the agar plates.

C. bombicola was first transferred from cryogenic storage (−80° C.) onto agar plates, and incubated at 25° C. for 48 hours. Single colonies from these plates were then used to inoculate 50 ml of medium in 250 ml shake flasks, which were incubated at 25° C. and 200 rpm for 30 hours. This inoculum was diluted to an optical density at 600 nm of 20 with fresh media and used to inoculate the fermenter.

Fermentations were run at 25° C., and dissolved oxygen was controlled to 30% by varying the stirrer speed, whilst maintaining a constant aeration rate of 1 l min⁻¹. Fermenter pH was controlled to a value of 3.5 by the addition of 3M sodium hydroxide.

For Examples 1 and 2 separation was run depending on production rate with the separation operated on a plurality of occasions during fermentation with pauses between those occasions of operation. When separation was performed sophorolipid rich fermentation broth was continuously circulated from the fermenter, through the separator and back around to the fermenter, being pumped in 8 mm external diameter silicon tubing of 1 mm wall thickness, using peristaltic pumps. The flow rate of broth into and out of the separator was controlled to around 1 ml s⁻¹ giving a residence time in the separating chamber of 76 s.

In the separating chamber the sophorolipid phase separated out from the broth due to differences in relative density. In Example 1 the sophorolipid phase separated towards the top of the separating chamber and in Example 2 it separated towards the bottom.

For both Example 1 and Example 2, during the separation operations, initially broth was continuously circulated and the sophorolipid phase accumulated in the separating chamber. When the sophorolipid phase accumulating in the separating chamber reached 50% of the height of the settling chamber, which typically occurred after around three minutes of operation, the outlet pump was started to continuously remove the sophorolipid product phase at a rate controlled between 0.5 and 2 ml min⁻¹, depending on the accumulation or reduction of the sophorolipid phase in the settling vessel.

The separation was run periodically in both Examples 1 and 2, with the majority of the available sophorolipid phase separated, and run again when sufficient sophorolipid phase had accumulated.

In the illustrated Examples, whilst the separator is designed for continuous operation at the scale used separation occurs at a rate of 30-150 times the production rate. For this reason, the separator was run intermittently. Using larger scale apparatus (not shown) the broth can be continuously circulated from the fermenter, through the separator and back around to the fermenter.

Separation was carried out at 111, 184 and 261 hours in Example 1, and 71.5, 281, 355 and 376 hours in Example 2, and no separation of the sophorolipid phase was carried out in Comparative Example 1C.

Analytical Techniques

For all analyses, 5 ml of broth was removed from the fermenter at each sample time point. The sample was centrifuged at 5000 rpm for 5 minutes using a Sigma 6-16S centrifuge (Sigma laboratory centrifuges, Germany) and the glucose in the supernatant quantified using a TrueResult® blood glucose monitor (Nipro Japan).

Residual oil and sophorolipid concentration were measured gravimetrically, with a hexane extraction first used to separate the residual oil and a triple ethyl acetate extraction used to separate the sophorolipid. The extracts were dried to constant weight in weighing dishes at ambient temperature for 30 h.

Cell growth was determined by both dry cell weight and optical density measurement. After solvent extraction, 8 ml distilled water was added to the remainder of the sample in the centrifuge tubes, which were then centrifuged at 8000 rpm for 10 minutes. The supernatant was discarded and the resulting cell pellet was resuspended in 8 ml distilled water. This cell suspension was transferred to drying trays, which were dried to constant weight at 90° C. in a drying oven. Optical density was used as a proxy for dry cell weight when diluting the inoculum, at a wavelength of 600 nm.

The structure of the sophorolipids produced was determined using negative ionisation electrospray ionisation, using an Agilent 6520 QTOF mass spectrometer (Agilent, United States). Samples were prepared by dissolving sophorolipid extracts in ethyl acetate, and filtering using a 0.2 μm filter. Flow injection analysis was used, at 0.3 ml min⁻¹, 50% acetonitrile, 0.1% formic acid, 49.9% water, with an injection volume of 2 μl.

Results

The separation parameters achieved in Examples 1 and 2 are shown in Table 1 and the key metrics for the Examples 1 and 2 and Comparative Example 1C are shown in Table 2.

TABLE 1
Separation parameters

| | | | | | |
|---|---|---|---|---|---|
| | | Sophorolipid | Sophorolipid | | |
| | Sophorolipid | concentration | concentration | | Recov- |
| Time | recovered | in fermenter | in extract | Enrich- | ery |
| (h) | (g) | (g l⁻¹) | (g l⁻¹) | ment | (%) |

Example 1

| | | | | | |
|---|---|---|---|---|---|
| 111 | 97.1 | 147.7 | 550.6 | 3.73 | 37 |
| 184 | 99.2 | 118.0 | 461.6 | 3.91 | 37 |
| 261 | 83.8 | 89.2 | 540.9 | 6.07 | 22 |
| total | 280.15 | | | | 86 |

Example 2

| | | | | | |
|---|---|---|---|---|---|
| 71.5 | 16.8 | 103.7 | 582.9 | 5.62 | 21 |
| 281 | 79.5 | 168.3 | 654.1 | 3.89 | 41 |
| 355 | 59.2 | 109.2 | 616.9 | 5.66 | 48 |
| 376 | 45.5 | 106.3 | 638.7 | 6.01 | 43 |
| total | 201.0 | | | | 57 |

TABLE 2
Key metrics
Example 1

| Yield substrate consumed (g g⁻¹) | Yield substrate fed (g g⁻¹) |
|---|---|
| 0.53 | 0.37 |

| Productivity starting volume | |
|---|---|
| (g l⁻¹ h⁻¹) | Productivity max volume (g l⁻¹ h⁻¹) |
| 1.07 | 0.69 |

Comparative Example 1C

| Yield substrate consumed (g g⁻¹) | Yield substrate fed (g g⁻¹) |
|---|---|
| 0.43 | 0.33 |

| Productivity starting volume | |
|---|---|
| (g l⁻¹ h⁻¹) | Productivity max volume (g l⁻¹ h⁻¹) |
| 1.07 | 0.62 |

Example 2

| Yield substrate consumed (g g⁻¹) | Yield substrate fed (g g⁻¹) |
|---|---|
| 0.42 | 0.39 |

| Productivity starting volume | |
|---|---|
| (g l⁻¹ h⁻¹) | Productivity max volume (g l⁻¹ h⁻¹) |
| 0.77 | 0.57 |

As can be seen from Table 1, the capacity to recover the majority of the sophorolipid from a fermentation broth during fermentation was demonstrated, whether the sophorolipid phase was less dense (Example 1) or denser (Example 2) than the fermentation broth.

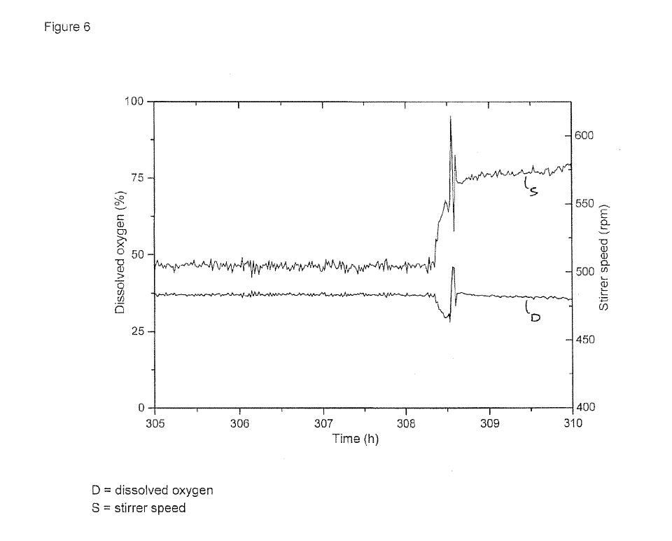

Sophorolipid recovery in Example 1 using separation resulted in a higher productivity than was achieved by Comparative Example 1C without the separation due to the reduced maximum fermenter volume. As shown by FIG. 6 which shows the effect of adding collected sophorolipids back to the broth of Example 1, using separation also provides for a lower agitation requirement to maintain the desired dissolved oxygen level.

For both Example 1 and Example 2, the majority of the sophorolipid was removed from the fermentation broth. Sophorolipid recovery was significantly higher for separation from the surface of the broth (Example 1), than from the bottom (Example 2), at 86% compared to 57%, but this may be largely due to the lower separation time for the final separation in Example 2.

In Examples 1 and 2 which were performed on a laboratory scale, the separation had to be stopped as the layer of sophorolipids at the bottom/top of the settling column became too low, to prevent the media and cell phase being entrained in the product stream. It is likely that with an increased scale, recoveries may be improved as the minimum sophorolipid phase depth, which must be recycled back to the fermenter, would be similar irrespective of fermenter volume.

Almost no cells or oil were removed by the separation in Example 2. For Example 1, cell removal was negligible. Whilst 68 g of oil was removed in Example 1, it may be possible to substantially reduce or eliminate that by better control of the oil feeding rates to maintain a low oil concentration in the fermenter.

The enrichment varied significantly between extractions at different time points, from 3.73 to 6.07. This is thought to be largely due to the sophorolipid concentration present in the fermenter before the separation, as there was little variation of the concentration in the extract, of approximately 550 g l⁻¹. It is likely that with an increased fermenter volume, the system may operate at lower initial sophorolipid concentrations and so may give an improved enrichment.

The total sophorolipid produced was calculated by adding the mass of sophorolipid in the fermenter and the mass of sophorolipid extracted from the fermenter. Substrate feeding meant the volume was higher than the 1 l starting volume during much of the fermentations.

For Example 1, the productivity at the maximum volume was 0.69 g l⁻¹ h⁻¹, and for Comparative Example 1C it was 0.62 g l⁻¹ h⁻¹, showing an effective productivity increase of 11% when using separation. This was due to the decreased maximal volume reached when separation was used, at 1540 ml with separation rather than 1720 ml without separation. The corresponding productivity for Example 2 of 0.57 g l⁻¹ h⁻¹ is not directly comparable due to the differences in feeding rates between the fermentations.

It is likely that using separation in continuous mode from early in a fermentation may make it possible to control the fermentation volume to around 1.3 times the initial volume (which without control may rise to around 1.7 times the initial volume after 280 hours). This may amount to an effective productivity increase of over 30%.

Sophorolipid was first extracted at 71.5 hours in Example 2, when a sophorolipid phase could be observed to settle in a sample bottle within 2 minutes. Settling then became ineffective until 283 hours due to the high residual glucose concentrations caused by pulse glucose feeding. Whilst the settling or floating of the sophorolipid also depends on other factors, a glucose concentration of 50 g l⁻¹ tends to represent a threshold of settling or floating to the surface. It is likely better control of the feeding rate may have enabled the sophorolipid to be settled throughout the fermentation.

In Example 1, the glucose concentration initially rose, and remained above 50 g l⁻¹ for the majority of the fermentation, which coupled with the significant residual rapeseed oil concentrations after around 140 hours led to the sophorolipid rising to the surface of the fermenter and forming a mixed phase with the residual oil, when oil was present in significant quantities.

Hydrodynamic and Mass Transfer Effects

The sophorolipid extracts from Example 1 were pooled after the fermentation, and returned to the fermenter at 308.3 h, over a period of 12 minutes. FIG. 6 shows the dissolved oxygen level and stirrer speed at the end of Example 1, with a drop in dissolved oxygen upon addition of the viscous sophorolipid phase. The presence of this sophorolipid phase effectively reduced the Kla in the fermenter, resulting in an increase in stirrer speed to maintain the dissolved oxygen at the setpoint. A stirring rate increase of around 75 rpm was required to maintain the desired dissolved oxygen level when the sophorolipid phase produced over the whole fermentation was added which resulted in a greater than 40% increase in stirring power requirement.

### Example 3

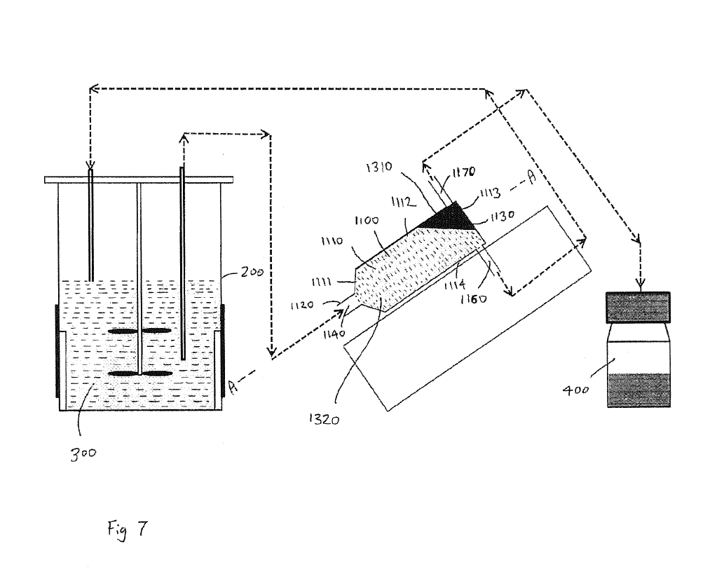

An alternative separator and fermentation conditions were used for Example 3. The separator used was an in house built settling column illustrated in FIG. 7.

The separator 1100 comprises a separating chamber 1110. At the first, lower, in use, end 1120 of the separator 1100 the separating chamber 1110 has a frusto conical section 1111. The separating chamber 1110 further comprises a cylindrical section 1112 having a diameter of 50 mm and a length of 150 mm. The base 1113 of the cylindrical section forms the upper, in use, end 1130 of the separator.

The separator 1100 comprises a broth inlet 1140 and has an opening to the separating chamber 1110 located on the longitudinal axis A-A of the separating chamber 1110 at the first end 1120 of the separator 1100. The inlet receives broth 300 comprising lipid product from a fermenter 200.

The separator comprises a first outlet (IIIA) shown as 1160 in FIG. 7 and a second outlet (IIIB) shown as 1170 in FIG. 7.

The first outlet 1160 is located toward the second end 1130 of the separator 1100 and has an opening to the separating chamber 1110 through a side wall 1114 of the separating chamber 1110. In use, the separator 1100 is oriented such that the first outlet 1160 lies below the longitudinal axis A-A of the separating chamber 1110. Depending on the density of a lipid phase separated from a broth in the separator 1100, in use, the first outlet 1160 can be used to transfer a lipid product from the separator 1100 or alternatively can be used to transfer a broth having had lipid product separated therefrom from the separator 1100. In Example 3 as illustrated by FIG. 7 the outlet 1160 was used to transfer a broth 1320 having had lipid product separated therefrom to the fermenter 200

The second outlet 1170 is located toward the second end 1130 of the separator 1100 and has an opening to the separating chamber 1110 through a side wall 1114 of the separating chamber 1110. In use, the separator 1100 is oriented such that the second outlet 1170 lies above the longitudinal axis A-A of the separating chamber 110. Depending on the density of a lipid phase separated from a broth in the separator 100, in use, the second outlet 1170 can be used to transfer a lipid product from the separator 1100 or alternatively can be used to transfer a broth having had lipid product separated therefrom from the separator 1100. In Example 3 as illustrated by FIG. 7 the outlet 1170 was used to transfer lipid product 1310 to a container 400.

The separating chamber 1110 is attached to a stand (not shown) which allows the angle of the longitudinal axis A-A of the separating chamber 1110 relative to the horizontal to be varied. For Example 3 the angle used was 30 degrees to the horizontal.

In Example 3, an increased media concentration, comprising 18 g l⁻¹ yeast extract and 15 g l⁻¹ peptone was used to achieve a high cell density and more rapid production. This necessitated higher feeding rates, as shown in FIG. 9. The stirring rate was fixed at 900 rpm, and the fermentation carried out in a 3 l volume Applikon bioreactor (fermenter), 1.5 l initial volume. The separator of FIG. 7 was used in Example 3 so as to achieve better separation of lipid product from the surface of the broth. All other parameters were as for Examples 1 and 2.

Higher cell densities resulted in a much faster sophorolipid production rate, and a total production of 689.8 g l⁻¹ sophorolipid was reached, The integrated separation system was necessary for extended running of the fermentation, to both prevent bioreactor overflow (which would have otherwise occurred around 165 h and 392 g l⁻¹ sophorolipid) and oxygen depletion, which dropped below the desired 30% set point within 100 hours and was corrected by separation.

This meant a productivity of 2.24 g l⁻¹ could be maintained for the whole fermentation, with no decrease in productivity apparent, other than at the point of additional nitrogen sources. These were added around 270 h, and resulted in a brief biomass growth period followed by continued production. It will be appreciated that this may be useful for periods of cell growth between long periods of sophorolipid production in continuous fermentation using this technique.

79.2% of the sophorolipid produced during the fermentation was recovered, i.e. a total of 819 g.

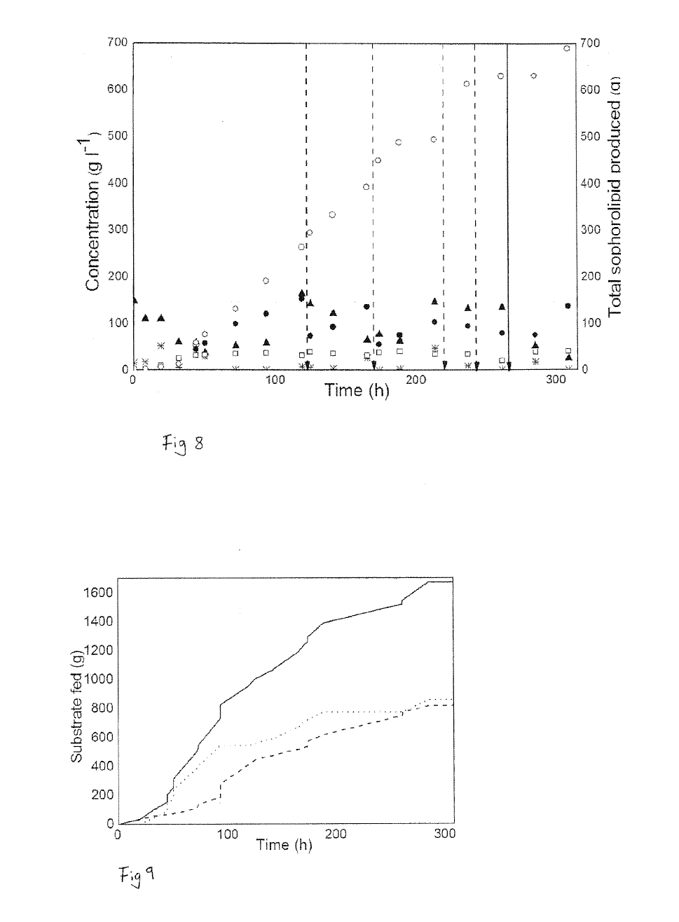

FIG. 8 illustrates the concentrations of substrates namely glucose (illustrated by filled triangles) and oil (indicated by stars) and dry cell weight (indicated by open squares), within the bioreactor and the production of sophorolipid (with the concentration of sophorolipid indicated by filled circles and the total sophorolipid produced indicated by open circles).

FIG. 9 illustrates the substrate feeding rate for oil (indicated by dashed line) glucose (indicated by dotted line) and total (indicated by solid line).

In FIG. 8 the arrows with dotted lines show the times when integrated sophorolipid separation was performed and solid lines show the addition of 12 g yeast extract and 10 g peptone dissolved in 100 ml water to the bioreactor.

It will be appreciated that preferred embodiments of the present invention may provide improved methods of sophorolipid production and in particular may allow for improved productivity and lower energy requirements.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. A method of producing lipids, wherein the method comprises:
(a) performing a fermentation in a fermenter to produce a broth comprising lipid product;
(b) transferring broth comprising lipid product from the fermenter to a separator;
(c) allowing a lipid phase comprising lipid product to separate from other constituents of the broth in the separator;
(d) returning broth having had lipid product separated therefrom from the separator to the fermenter; and
(e) transferring lipid product from the separator.

2-30. (canceled)

31. The method according to claim 1, wherein the method comprises producing lipids selected from the group consisting of hydrocarbons, terpenoids, fats, oils, fatty acids and glycolipids, preferably terpenoids, fats, oils, fatty acids and glycolipids.

32. The method according to claim 1, wherein the lipid product is selected from sophorolipids, rhamnolipids and mannosylerythritol lipids.

33. The method according to claim 32, wherein a gravity separator is the only separator used within the method in step c).

34. The method according to claim 33, wherein the method comprises circulating broth between the fermenter and gravity separator, preferably continuously, over a period of at least 50 hours or over the duration of the production phase of the fermentation, wherein the method comprises transferring broth from the fermenter and returning broth to the fermenter such that the broth in the fermenter comprises at least 80% by weight, preferably at least 90% by weight, most preferably at least 95% by weight of the sum of the broth in the fermenter and gravity separator; and wherein the method comprises adding substrate to the fermenter over the duration of the production phase of the fermentation.

35. The method according to claim 1, wherein the method comprises maintaining the concentration of lipids in the broth in the fermenter below 80 g·L⁻¹ to prevent bioreactor overflow and dissolved oxygen depletion.

36. The method according to claim 1, wherein the broth is agitated to prevent phase separation in the fermenter, and substantially no phase separation occurs in the fermenter.

37. The method according to claim 1, wherein the gravity separator comprises a separating chamber and is adapted such that, in use, the longitudinal axis of the chamber lies at between 10 degrees and 60 degrees to the horizontal.

38. The method according to claim 1, wherein step (c) comprises allowing separation of the broth by gravity in a gravity separator to provide a lipid phase comprising lipid product and a bulk broth comprising other constituents of the broth and wherein the bulk broth in the gravity separator comprises substrate and/or cells and wherein the method comprises separating the lipid phase comprising substantially no cells; and wherein the lipid phase comprises lipid product and water in a concentration of at least 80% by weight, preferably at least 90% by weight, most preferably at least 95% by weight.

39. The method according to claim 33, wherein a sophorolipid product is generated by controlling fermentation conditions, wherein the pH is between 2-5, a sugar is fed at a rate of at least 0.5 g·L⁻¹·h⁻¹, a vegetable oil is fed at a rate of at least 0.5 g·L⁻¹·h⁻¹ and a dissolved oxygen level of the broth in the fermenter is at least 20%.

40. A gravity separator adapted to separate a lipid phase from other constituents of a broth comprising lipids, wherein the gravity separator comprises:
(I) a separating chamber in which, in use, said broth can be allowed to reside for a period of time such that a lipid phase comprising lipid product separates from other constituents of the broth; and wherein optionally the separating chamber is adapted such that, in use, a longitudinal axis of the chamber lies at between 10 degrees and 60 degrees to the horizontal;
(II) a broth inlet to the separating chamber for transferring broth comprising lipid product into the separating chamber, in use; and
(III) outlets from the separating chamber for: (i) transferring broth having had lipid product separated therefrom from the separating chamber, in use; and (ii) transferring lipid product from the separating chamber, in use.

41. The gravity separator according to claim 40, wherein the gravity separator comprises three outlets (III):
(IIIa) a first outlet located toward the upper, in use, end of the separating chamber;
(IIIb) a second outlet located toward the lower, in use, end of the separating chamber; and
(IIIc) a third outlet located toward the lower, in use, end of the separating chamber.

42. The gravity separator according to claim 41, wherein outlets (IIIa), (IIIb) and (IIIc) are configured, to be selectively used as follows:
(1) (IIIa) used for pressure relief,
(IIIb) for transferring a lipid product from the separating chamber,
(IIIc) for transferring broth having had a lipid product removed therefrom from the separating chamber; or
(2) (IIIa) for transferring a lipid product from the separating chamber;
(IIIb) for transferring broth having had a lipid product removed therefrom from the separating chamber;
(IIIc) not used.

43. The gravity separator according to claim 40, wherein the gravity separator comprises:
(I) a separating chamber in which, in use, said broth can be allowed to reside for a period of time such that a lipid phase comprising lipid product separates from other constituents of the broth;
(II) a broth inlet which located toward a first end of the separating chamber for transferring broth comprising lipid product into the separating chamber, in use;
(IIIA) a first outlet located toward a second end of the separating chamber; and
(IIIB) a second outlet located toward a second end of the separating chamber; and wherein the broth inlet (II) comprises an opening to the separating chamber at a lower, in use, end of said separating chamber which lies on the longitudinal axis of said separating chamber; the first outlet (IIIA) comprises an opening to the separating chamber which lies in a side wall of said separating chamber in a region of said side wall towards an upper, in use, end of the separating chamber and wherein said opening to the separating chamber lies below the longitudinal axis of said separating chamber, in use; and the second outlet (IIIB) comprises an opening to the separating chamber which lies in a side wall of said separating chamber in a region of said side wall toward the upper, in use, end of the separating chamber and wherein said opening to the separating chamber lies above the longitudinal axis of said separating chamber, in use.

44. An apparatus for producing lipids, said apparatus comprising a fermenter having a fermentation chamber and the gravity separator according to claim 40, wherein the fermentation chamber and separating chamber of the gravity separator are in fluid communication such that, in use, broth comprising lipid product can be transferred from the fermentation chamber to the separating chamber and broth having had lipid product separated therefrom can be transferred from the separating chamber to the fermentation chamber; and wherein optionally the separating chamber is adapted such that, in use, the longitudinal axis of the chamber lies at between 10 degrees and 60 degrees to the horizontal.
